(19) United States
(12) Patent Application Publication
(10) Pub. No.: US 2007/0093518 A1
(43) Pub. Date: Apr. 26, 2007

(54) Pharmaceutical compositions for the treatment of organophosphate poisoning

(76) Inventors: Janet Ruth Wetherell, SALISBURY, WILTSHIRE (GB); Matthew Edward Price, Salisbury (GB); Helen Mumford, Salisbury (GB)

Correspondence Address: NIXON & VANDERHYE, PC, 901 NORTH GLEBE ROAD, 11TH FLOOR, ARLINGTON, VA, 22203, US

(21) Appl. No.: 10/572,307

(22) PCT Filed: Sep. 14, 2004
(86) PCT No.: PCT/GB04/03913

§ 371 Date: Apr. 6, 2006

(30) Foreign Application Priority Data
Sep. 19, 2003 (GB) 0321976.3
Feb. 20, 2004 (GB) 0403714.9

(51) Int. Cl. A61K 31/473; A61K 31/46; A61K 31/444; A61K 31/403

(52) U.S. Cl. 514/290; 514/304; 514/411; 514/316; 514/358; 514/332; 514/357

(57) ABSTRACT

A pharmaceutical composition comprising (i) a carbamate; (ii) a first anticholinergic; (iii) a second anticholinergic; and (iv) a pyridinium salt. The pharmaceutical composition is suitable for the treatment of organophosphate poisoning, including nerve agent and pesticide poisoning. The pharmaceutical composition provides an effective therapy against the lethal effects of nerve agent, provides effective protection from the effects of incapacitation and does not require the use of a pre-treatment. A kit comprising the pharmaceutical composition is also described and claimed.

This invention relates to pharmaceutical compositions and their use for the treatment of organophosphate poisoning.

Organophosphates are a class of highly toxic compounds that include pesticides and nerve agents. Nerve agents pose a risk to both military and civilian populations due to their potential use as chemical warfare agents, and more recently for their potential use in terrorism. There is also a growing concern over the widespread use of organophosphate pesticides. These pesticides are used in agriculture, homes, gardens and in veterinary practices and all of them exhibit some level of toxicity to humans. Both types of organophosphate act by irreversibly inhibiting peripheral and central acetylcholinesterase (AChE), which results in a rapid accumulation of acetylcholine causing over-stimulation of muscarinic and nicotinic receptors. The signs of poisoning include hypersecretion, convulsions, respiratory distress, coma and death. In order to treat and protect those at risk from organophosphate poisoning, it is necessary to identify and develop suitable medical countermeasures.

Those at risk can be protected from the effects of nerve agents by a combination of pretreatment and immediate therapy. For example, individuals are issued with a pyridostigmine pretreatment, which is supported by post-exposure treatment with a drug combination of atropine sulphate (a muscarinic antagonist), pralidoxime mesylate (P2S; an pyridinium oxime) and avizafone (a water-soluble pro-drug of the anticonvulsant diazepam) achieved with up to three doses from the ComboPen® autoinjector (Meridian Medical Technologies Inc., Maryland, USA). This drug combination has been shown to protect guinea-pigs (Leadbeater et al., Fund. Appl. Toxicol. 5 (1985), S225-231) and non-human primates (Dirnhuber et al., J. Pharm. Pharmacol. 31 (1979)295-299) against the lethal effects of nerve agent poisoning but it is less effective against the incapacitation that is observed due to the poisoning.

A significant problem with the current approach, and in general with the use of pretreatments, is that there are situations where the administration of a pretreatment is not possible or is impractical. Although it may be possible to administer a pretreatment to an entire Regiment under a nerve agent threat, it is highly impractical to administer the same pretreatment to a large portion of the general public during a perceived terrorism threat or to those at risk of pesticide poisoning. In addition, pretreatments are generally given to healthy (unpoisoned) individuals and may themselves produce side effects, for example, pyridostigmine acts by inhibiting a portion of peripheral cholinesterase, thus protecting it from irreversible inhibition by the nerve agent. Therefore the pyridostigmine must be administered at a controlled dose to ensure that protection is afforded without impairing performance.

There remains a need to identify further methods of treating organophosphate poisoning. Ideally such a treatment would be equally if not more effective than the current treatment in protecting against the lethal effects of nerve agents, but would show improved protection against the effects of incapacitation. It would be ideal if a treatment could be developed that can be administered as a single-dose therapy without the requirement for a pretreatment.

A new therapy has now been developed, which has been shown to be effective in the treatment of organophosphate poisoning. The therapy comprises a drug combination that may be administered as a single dose, which does not require the use of a pretreatment. Experimental results have been obtained by testing the new therapy against nerve agent poisoning in the Dunkin-Hartley guinea pig. These results show that the new therapy protects against the lethal effects of nerve agents, including GA and GD, and also minimises incapacitation following poisoning. Furthermore, the results suggest that the drug combination is likely to be effective against poisoning from a wider range of organophosphate compounds, including pesticides.

This new therapy has several advantages. The new drug combination is at least as effective as the current treatment in protecting against the lethal effects of nerve agents and provides greater protection against the effects of incapacitation. The treatment can administered as a single-dose therapy and has the added advantage that it can be administered without the requirement of a pretreatment.

It is an object of this invention to identify a new pharmaceutical composition for the treatment of poisoning by organophosphates, including nerve agents and pesticides. It is another object of this invention that the pharmaceutical composition provides protection from the lethal effects of nerve agent poisoning. It is an additional object of this invention that the pharmaceutical composition provides improved protection from the incapacitating effects of nerve agent poisoning. It is also an object of this invention to provide a pharmaceutical treatment that can be administered as a single dose therapy. It is a further object of this invention to provide a pharmaceutical treatment that does not require the use of a pretreatment. It is yet another object of this invention to provide a method for the treatment of organophosphate poisoning in a mammal, including man, comprising administration of an effective amount of a pharmaceutical composition. These, and other objects of this invention, will become apparent in light of the following disclosure.

### SUMMARY OF THE INVENTION

According to a first aspect this invention relates to a pharmaceutical composition comprising
(i) a carbamate;
(ii) a first anticholinergic,
(iii) a second anticholinergic, and
(iv) a pyridinium salt.

According to a second aspect this invention relates to a kit comprising
(i) a carbamate;
(ii) a first anticholinergic,
(iii) a second anticholinergic, and
(iv) a pyridinium salt.
   wherein each component is dosed simultaneously, sequentially or separately.

According to a third aspect this invention relates to the use of a pharmaceutical composition comprising
(i) a carbamate;

(ii) a first anticholinergic,
(iii) a second anticholinergic, and
(iv) a pyridinium salt.
   for the manufacture of a medicament for the treatment of poisoning by organophosphates, including nerve agents and pesticides.

According to a fourth aspect this invention relates to a method for the treatment of organophosphate poisoning in a mammal, including man, comprising administration of an effective amount of a pharmaceutical composition comprising
(i) a carbamate;
(ii) a first anticholinergic,
(iii) a second anticholinergic, and
(iv) a pyridinium salt.

### DETAILED DESCRIPTION OF THE INVENTION

All publications cited herein are hereby incorporated by reference in their entirety, unless otherwise indicated.

The elements of the invention are described in more detail in below.

Some of the compounds of the present invention may comprise asymmetrically substituted carbon atoms. Such asymmetrically substituted carbon atoms can result in medicaments of the present invention comprising mixtures of stereoisomers at a particular asymmetrically substituted carbon atom or single stereoisomer. As a result, racemic mixtures, mixtures of diastereoisomers, as well as single diastereoisomers of the compounds of the invention are included in the present invention.

This invention relates to a pharmaceutical composition comprising
(i) a carbamate;
(ii) a first anticholinergic,
(iii) a second anticholinergic, and
(iv) a pyridinium salt.

As used herein the term “carbamate” means derivatives of carbamic acid, including salts and esters, including urethanes (ethyl esters of carbamic acid). The carbamate can be any such derivative that slows down the breakdown of acetylcholine by partial inhibition of acetylcholinesterase. It is preferred that the carbamate is a carbamic ester or a pharmaceutically acceptable salt thereof. Such esters include, but are not limited to; rivastigmine; neostigmine; pyridostigmine; physostigmine; derivatives of physostigmine such as those described in U.S. Pat. No. 5,081,117; thiaphysovenine and analogues thereof (as described in U.S. Pat. No. 5,378,723); phenserine and analogues thereof (as described in U.S. Pat. No. 5,171,750) and pharmaceutically acceptable salts thereof. It is preferred that the carbanate is physostigmine or a derivative thereof Suitable carbamates also include metabolites of carbamates, such as norphysostigmine and other metabolites which slow down the breakdown of acetylcholine and pharmaceutically acceptable salts thereof It is most preferred that the carbamate is physostigmine salicylate.

As used herein the term “anticholinergic” means any chemical, drug or drug effect that causes partial or total blockage of the action of the neurotransmitter acetylcholine.

It is preferred that the first anticholinergic of the pharmaceutical composition of the present invention is a cholinomimetic alkaloid that exhibits an antinicotinic and/or antimuscarinic effect. Suitable anticholinergics include, but are not limited to, anisotropine, atropine, belladonna, clinidiun, dicyclomine, glycopyrrolate, homatropine, hyoscyamine, mepenzolate, methantheline, methscopolamine, pirenzepine, propantheline and hyoscine (also known as scopolamine). It is preferred that the anticholinergic is selected from the group consisting of aprophen, atropine, azaprophen, benactyzine, biperiden, procyclidine, hyoscine and pharmaceutically acceptable salts thereof. It is more preferred that the anticholinergic is a pharmaceutically acceptable salt of hyoscine, such as hyoscine hydrobromide.

The second anticholinergic can be any of those listed above for the first anticholinergic. It is preferred that the second anticholinergic is selected from the group consisiting of aprophen, atropine, azaprophen, benactyzine, biperiden, procyclidine, hyoscine and pharmaceutically acceptable salts thereof, such that the second anticholinergic is different to the first. The first and second anticholinergic can be different salts of the same anticholinergic. It is more preferred that the second anticholinergic is a pharmaceutically acceptable salt of hyoscine. It is even more preferred that, when the first anticholinergic is hyoscine hydrobromide, the second anticholinergic is hyoscine methyl nitrate.

The pyridinium salt of the pharmaceutical composition of the present invention is preferably a pyridinium oxime of general formula: 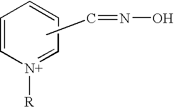
wherein R is selected from the group consisting of hydrogen, loweralkyl, alkoxy, alkenyl, alkynyl, aryl, aryloxy, cycloalkyl, halogen, haloalkyl, amino, imino, nitro, cyano, carbamyl, formyl, pyridinium, alkyl-pyridinium and alkoxy-pyridinium.

It is preferred that the pyridinium salt is a mono or bis-pyridinium compound that contains at least one oxime functional group or is the de-oximninomethyl analogue of such an oxime. It is more preferred that the pyridinium salt is selected from the group consisting of compounds according to formula I: 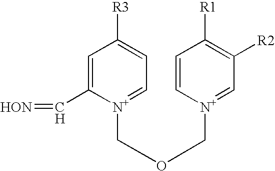
and pharmaceutically acceptable derivatives and pharmaceutically acceptable salts thereof, wherein R1 is selected from the group consisting of hydrogen and —CONH₂; wherein R2 is selected from the group consisting of hydrogen, —CONH₂, —COC₆H₅ and —COC₆H₁₁ and wherein R3 is selected from the group consisting of hydrogen and —CHNOH;
a compound according to formula II: 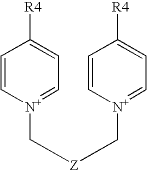
and pharmaceutically acceptable salts thereof, wherein when Z is oxygen, R4 is selected from the group consisting of hydrogen, —CHNOH and —C(CH₃)₃ and wherein when Z is —CH₂—, R4 is selected from the group consisting of hydrogen and —CHNOH;
a compound according to formula III: 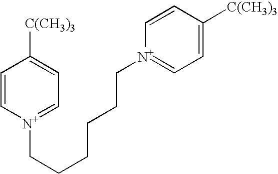
and pharmaceutically acceptable derivatives and pharmaceutically acceptable salts thereof;
a compound according to formula IV: 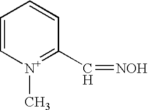
and pharmaceutically acceptable derivatives and pharmaceutically acceptable salts thereof;
and mixtures thereof

It is more preferred that the pyridinium salt is a bis-pyridyl oxime compound according to formula I wherein R1, R2 and R3 are all hydrogen or where R1 is hydrogen, R2 is —CONH₂ and R3 is hydrogen or where R1 is CONH₂ and R2 and R3 are hydrogen. Examples of suitable compounds, which may be readily synthesized by one skilled in the art include, but are not limited to, those described in French, M. C., Wetherell, J. R. and White, P. D. T. Eur. J. Pharmacol. 91 (1983) 399, especially pharmaceutically acceptable salts of the 1-(2′-hydroxyiminomethyl-1′-pyridinium)-3-(1″-pyridinium)-2-oxapropane dication (commonly known in the art as HS14), and the (1-(2′-hydroxyiminomethyl-1′-pyridinium)-3-(3″carbamoyl-1″-pyridinium)-2-oxapropane dication (commonly known in the art as HS6) and its structural isomer, the (1-(2′-hydroxyiminomethyl-1′-pyridinium)-3-(4″carbamoyl-1″-pyridinium)-2-oxapropane dication (commonly known in the art as HI6). It is even more preferred that the pyridinium salt is a pharmaceutically acceptable salt of HI6.

It is preferred that the weight ratio of (i):(ii) is in the range of from about 1:5 to about 1:20; that the weight ratio of (i):(iii) is in the range of from about 1:5 to about 1:20; that the weight ratio of (i):(iv) is in the range of from about 1:200 to about 1:800; that the weight ratio of (ii):(iii) is in the range of from about 1:2 to about 2:1; that the weight ratio of (ii):(iv) is in the range of from about 1:20 to about 1:80 and that the weight ratio of (iii): (iv) is in the range of from about 1:20 to about 1:80. It is more preferred that the ratio of (i):(ii):(iii):(iv) is about 1:10:10:500.

The pharmaceutical composition of the present invention may additionally comprise a fifth pharmaceutical compound, hereinafter called a pharmaceutical adjunct, such as a second pyridinium salt. Examples of suitable pharmaceutical adjuncts include, but are not limited to, any of those pyridinium salts listed above. It is preferred that the pharmaceutical adjunct is a pharmaceutically acceptable salt of a bis-pyridinium oxime according to Formula II, wherein when Z is oxygen, R4 is selected from the group consisting of hydrogen, —CHNOH and —C(CH₃)₃. It is more preferred that the pharmaceutical adjunct is a pharmacuetically acceptable salt of toxogonin. It is also preferred that the pharmaceutical adjunct is a different pyridinium salt to the pyridinium salt of the pharmaceutical composition. It is more preferred that when the pyridinium salt of the pharmaceutical composition is HI-6, the pharmaceutical adjunct is toxogonin.

It is preferred that the weight ratio of pharmaceutical adjunct to pharmaceutical composition is in the range of 1:4 to 1:20. It is more preferred that the weight ratio of pharmaceutical adjunct to pharmaceutical composition is in the range of 1:6 to 1:12, even more preferably in the range of about 1:8.

In order to prepare the pharmacuetical composition of the present invention it may be necessary to use the compounds in the form of salts derived from inorganic or organic acids. Such salts can lead to materials that are either water soluble, oil soluble or dispersible as is desired by the formulator. Organic salts include but are not limited to the following: acetate, adipate, alginate, aspartate, benzoate, benzenesulfonate, bisulfate, butyrate, camphorate, camphorsulphonte, citrate, carbonate, digluconate, cyclopentanepropionate, dodecylsulphate, ethanesulphonate, glucoheptanoate, glycerophosphate, hemisulphate, heptanoate, hexanoate, fumarate, 2-hydroxyethanesulphonate, lactate, maleate, methanesulphonate, nicotinate, 2-napthalenesulphonate, oxalate, pamoate, pectinate, persulphate, 3-phenylproionate, picrate, pivalate, propionate, salicylate, succinate, tartrate, thiocyanate, p-toluenesulphonate and undecanoate. Examples of inorganic acids which may be employed to form pharmaceutically acceptable salts include hydrochloric acid, hydrobromic acid, hydroiodic acid, sulphuric acid, phosphoric acid. Pharmaceutically acceptable salts include but are not limited to cations based on the alkali and alkaline earth metals such as sodium, lithium, potassium, calcium, magnesium, aluminium salts and the like. In addition the basic nitrogen-containing groups can be quarternised with such agents as loweralkyl halides such as methyl, ethyl, propyl and butyl chloride, bromides and iodides; dialkyl sulphates like dimethyl, diethyl, dibutyl and diamyl sulphates, long chain halides such as decyl, lauryl, myristyl and stearyl chlorides, bromides and iodides, aralkyl halides like benzyl and phenethyl bromides and others. Preferred examples of quarternising agents include ammonia, or an organic primary, secondary or tertiary amine.

This invention also relates to a kit of parts comprising
(i) a carbamate;
(ii) a first anticholinergic,
(iii) a second anticholinergic, and
(iv) a pyridinium salt.
   wherein the four components are packaged such that they may be dosed simultaneously, sequentially or separately.

In a further embodiment the kit comprises
(i) a pyridinium salt; and
(ii) a pharmaceutical composition comprising a first anticholinergic, a second anticholinergic and a carbamate
   wherein (i) and (ii) are dosed simultaneously, sequentially or separately.

In another embodiment, the kit additionally comprises a pharmaceutical adjunct, which may expedite or improve the action of the pharmaceutical composition, when added simultaneously, sequentially or separately. If the pharmaceutical adjunct is added separately to the pharmaceutical composition, it is preferred that it is added within 6 hours of the administration of the pharmaceutical composition. It is more preferred that the adjucnt is added within 4 hours and even more preferred that it is added within 2 hours of administration of the pharmaceutical composition. The pharmaceutical adjunct can be any of those pyridinium salts described above but it is preferred that it is a pharmaceutical salt of toxogonin.

In each of these embodiments relating to the pharmaceutical composition and the kit, it is preferred that the components are formulated in a suitable form, such as a solid dose, a liquid dose or an injectable preparation.

Solid dosage forms for oral administration may include capsules, tablets, pills, powders and granules. In such solid dosage forms, the active components may be mixed with at least one inert diluent such as sucrose, lactose or starch or mixtures thereof. Such dosage forms may also comprise, as is normal practice, additional substances other than inert diluents e.g. lubricating agents, such as magnesium stearate. In the case of capsules, tablets and pills the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants such as wetting agents, emulsifying and suspending agents, and sweetening, flavouring and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a non-toxic parenterally acceptable diluent or solvent, for example, as a solution in ⅓ propandiol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The pharmaceutical compositions may also comprise other ingredients necessary to achieve optimum formulation and these can include, but are not limited to, preservatives, stabilisers, excipients, and natural or synthetic lecithins. The compositions of the present invention may also comprise liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolisable lipid capable of forming liposomes can be used.

It is preferred that the pharmaceutical composition is formulated as an injectable preparation.

This invention also relates to the use of the pharmaceutical compositions for the manufacture of a medicament for the treatment of organophosphate poisoning, including nerve agent and pesticide poisoning. The pharmaceutical compositions can be administered for the treatment of poisoning by organophosphates by any appropriate route. The method of administration will depend on several factors including the nature of the formulation that has been manufactured to contain the composition, the type and severity of poisoning, the patient and the like. The treatment can be administered by any medically appropriate route such that it results in an efficacious treatment for the type of poisoning that has occurred. For example, the treatment can be administered via intramuscular or intravenous injection to obtain a rapid response in the case of nerve agent poisoning. As an alternative example, the treatment can be administered orally, intradermally or intranasally, which may be more appropriate for less severe types of organophosphate poisoning, such as from pesticides. It is preferred that the pharmaceutical composition is administered intravenously or intramuscularly to obtain the most effective and/or rapid response. It is more preferred that the pharmaceutical composition is administered intramuscularly.

According to another aspect this invention relates to a method for the treatment of organophosphate poisoning in a mammal, including man, comprising administration of an effective amount of such a pharmaceutical composition. It will be understood that the specific dose for any particular patient will depend upon a variety of factors include the activity of the compound employed, age, body weight, health, sex, diet, time of administration, route of administration, rate of excretion, drug combination and the severity of the disease state.

It is preferred that the method of use comprises administering the first dose of treatment compound to the patient immediately after exposure of the patient to the source of organophosphate poison or immediately upon the first signs of poisoning. It is more preferred that the treatment should be administered within 10 minutes of exposure, preferably within 5 minutes, more preferably within 3 minutes, even more preferably within 2 minutes and most preferably within 1 minute. The length of time between exposure and administration of the treatment may depend on whether or not the patient had been treated with a pretreatment for organophosphate poisoning. However, the treatment may be used irrespective of whether or not any pretreatment had been administered prior to exposure. In particular, when the treatment is used in conjunction with a pretreatment the effects of both are additive, for example, a person who has received a pretreatment will exhibit less severe signs of poisoning and will recover more quickly upon administration of the therapy.

If a pharmaceutical adjunct is administered separately from the pharmaceutical composition, it is preferred that the adjunct is added within 6 hours, preferably within 4 hours and more preferably within 2 hours of administration of the pharmaceutical composition.

The invention will now be particularly described by way of example with reference to the accompanying diagrammatic drawings in which:

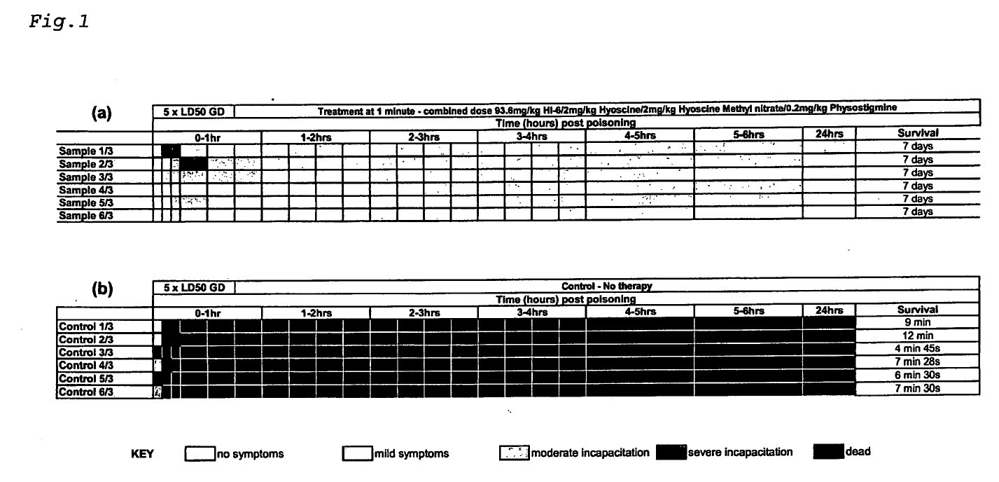

FIG. 1 illustrates the effects on guinea-pigs after exposure to the nerve agent GD. FIG. 1(a) shows the effects of the nerve agent on the guinea-pig over a 24 hour period, after treatment with a pharmaceutical composition, which was administered one minute after exposure to the nerve agent and record survival time up to seven days post-poisoning. FIG. 1(b) shows the effects of the nerve agent on control samples over the same time period, where no therapy was administered and records the survival time of each control sample.

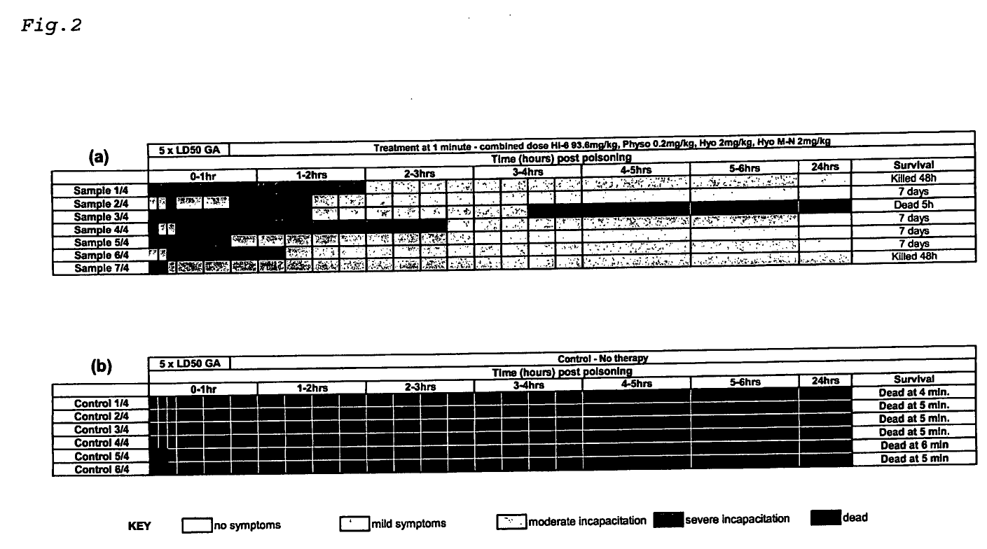

FIG. 2 illustrates the effects on guinea-pigs after exposure to the nerve agent GA. FIG. 2(a) shows the effects of the nerve agent on the guinea-pig over a 24 hour period, after treatment with the same pharmaceutical composition, also administered one minute after exposure to the nerve agent and records survival time up to seven days post-poisoning. As above FIG. 2(b) shows the effects of the nerve agent on control samples over the same time period, where no therapy was administered.

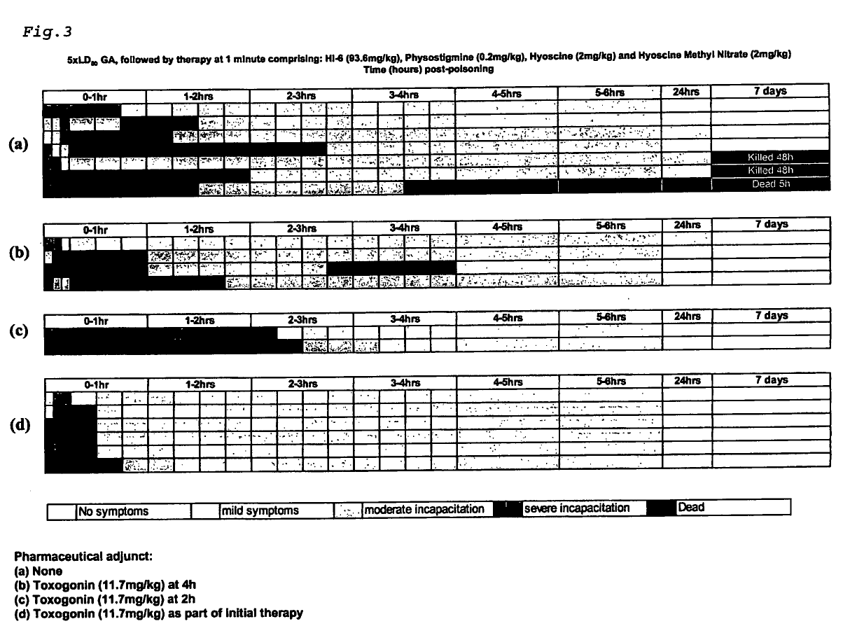

FIG. 3 illustrates the effects on guinea-pigs after exposure to the nerve agent GA. FIG. 3(a) shows the effects of the nerve agent on the guinea-pig over a 24 hour period, after treatment with a pharmaceutical composition, which was administered one minute after exposure to the nerve agent and records survival time up to seven days post-poisoning. FIG. 3(b) shows the effects on the guinea pig when the treatment additionally comprises administration of the pharmaceutical adjunct, toxogonin, at approximately 4 hours post-exposure. FIG. 3(c) shows the effects when the same pharmaceutical adjunct is administered at approximately 2 hours post-exposure and FIG. 3(d) shows the effects when the pharmaceutical adjunct is added as part of the initial therapy.

### EXAMPLES

These examples further illustrate the preferred embodiments within the scope of the present invention. These examples are given solely for the purpose of illustration and are not to be construed as limitations of the present invention as many variations of the invention are possible without departing from its spirit or scope.

Material and Methods

All experiments were performed according to the conditions of a project license issued under the Animals (Scientific Procedures) Acts, 1986.

Male Dunkin-Hartley guinea pigs (300-350 g) were allowed free access to food pellets and water and were given a small amount of hay. Thirteen days prior to nerve agent challenge, all animals were implanted subcutaneously with an identification/temperature transponder (Biomedic Data Systems Inc. USA) with local anaesthesia (0.07 ml of 2% xylocaine s.c.). The temperature and weight of each animal was measured daily as an indicator of general good health. The animals were single housed in the experimental room 5 days prior to nerve agent administration. The room lighting was 300 lux on a 24 h light:dark cycle (lights on 0600-1800 h), the room temperature was 18-20° C. and the humidity was 30-50%.

The organophosphorus nerve agents pinacolyl methyl phosphonofluoridate (GD) and dimethyl phosphonocyanidate (GA) were prepared to approximately 95% purity at the Defence Science and Technology Laboratory (Dstl), (Porton Down, UK) using published methods (see for example chapter 10 in “The Chemistry of Organophosphorus Chemical Warfare Agents”, Vol. 4, Ed F. R. Hartley, John Wiley, 1996) and stored as approximately 5.0 mg/ml solutions in isopropanol. Physostigmine salicylate, hyoscine hydrobromide and hyoscine methyl-nitrate were obtained from Sigma. HI6 ((1-(2′-hydroxyiminomethyl-1′-pyridinium)-3-(4″carbamoyl-1″-pyridinium)-2-oxapropane), as the dichloride salt, was donated as a gift from DRDC (Defence Research and Development, Suffield, Canada). The nerve agents and the drugs were made up to the appropriate concentrations in physiological saline (0.9%).

Guinea-pigs were challenged sub-cutaneously with 5×LD50 of GD (135 μg/kg) and GA (625 μg/kg). The therapy drugs were administered intramuscularly (0.33 ml/kg) as a combined single injection at 1 minute post-poisoning. The animals were closely observed for up to 24 hours and any observed symptoms characterised as:
(a)—no symptoms
(b)—mild symptoms
(c)—moderate incapacitation
(d)—severe incapacitation
(e)—dead

At 5, 6 and 24 hours post-poisoning, all animals were checked by weight and temperature. Any animals which exhibited a weight loss of greater than 20%, low temperature or which were still exhibiting clinical signs of intoxication were killed (commonly defined as the “humane endpoint”). Animals that were deemed healthy after 24 hours were monitored for a further 7 days. Control samples, where the guinea pig received no treatment, were used for each experiment and was subject to exactly the same conditions and observations as described above for the test samples.

### Example 1

Using the above protocol, guinea pigs were challenged with 5×LD50 of GD and treated after 1 minute with a single dose comprising physostigrnine salicylate (0.2 mg/kg), hyoscine hydrobromide (2 mg/kg), hyoscine methyl nitrate (2 mg/kg) and HI-6 (dichloride salt) (93.6 mg/kg).

Results

All of the six animals that received treatment 1 minute post GD poisoning developed mild or moderate symptoms within five minutes (FIG. 1a). In two cases this deteriorated to severe incapacitation after 30 minutes. However, after 1 hour all six animals displayed only mild symptoms, with 5 of the 6 showing no symptoms after 24 hours. All six animals were observed healthy after 7 days.

Six control samples were used in this experiment (FIG. 1b). All exhibited severe incapacitation within 5 minutes post-poisoning, with no animals surviving beyond 12 minutes.

### Example 2

Using the above protocol, guinea pigs were challenged with 5×LD50 of GA and treated after 1 minute with a single dose comprising physostigmine salicylate (0.2 mg/kg), hyoscine hydrobromide (2 mg/kg), hyoscine methyl nitrate (2 mg/kg) and HI-6 (dichloride salt) (93.6 mg/kg).

Results

All seven samples treated at one minute post GA poisoning showed severe incapacitation within 1 hour (FIG. 2a). All seven improved to show only moderate symptoms within 3 hours. One animal died at 5 hours, but the other 6 continued to improve and were displaying only mild or moderate symptoms at 24 hours. Of the six animals remaining at 24 hours, 2 were culled after 48 hours as the humane endpoint was reached. The remaining four animals were observed to be healthy after 7 days.

Six control samples were used (FIG. 2b). Severe incapacitation or death was observed for all animals within 5 minutes, with no survivors remaining beyond 6 minutes.

### Example 3

Using the above protocol, four sets of guinea pigs were challenged with 5×LD50 of GA and treated after 1 minute with a single dose comprising physostigrnine salicylate (0.2 mg/kg), hyoscine hydrobromide (2 mg/kg), hyoscine methyl nitrate (2 mg/kg) and HI-6 (dichloride salt) (93.6 mg/kg). In addition two of the sets were treated with the pharmaceutical adjunct, toxogonin (1 1.7mg/kg) at 4 hours and 2 hours post exposure. The fourth set of guinea pigs was treated with 11.7 mg/kg at the same time as the initial therapy.

Results

All samples treated at one minute post GA poisoning showed severe incapacitation within 1 hour (FIGS. 3a-d). The set of samples which received no pharmaceutical adjunct recovered to show moderate symptoms within 3-6 hours (FIG. 3a). One animal died at 4 hours, and another 2 died or were killed after 24 hours. The remaining four animals were observed to be healthy after 7 days. Those animals which received toxogonin after 4 hours showed similar recovery time (FIG. 3b), showing mild to moderate symptoms after 4-6 hours. No samples died or were culled and all were observed as healthy after 7 days. FIG. 3(b) shows two animals which received the pharmaceutical adjunct after 2 hours. By 4 hours both showed only mild symptoms and were fully recovered within 24 hours. FIG. 3(c) shows samples which received toxogonin as part of the initial therapy and all animals displayed only mild symptoms after 2 hours. All animals were fully recovered by 24 hours.

1) A pharmaceutical composition comprising
(i) a carbamate;
(ii) a first anticholinergic,
(iii) a second anticholinergic, and
(iv) a pyridinium salt.

2) A pharmaceutical composition according to claim 1 wherein the carbamate is selected from the group consisting of rivastigmine, neostigmine, pyridostigmine, physostigmine, phenserine, derivatives thereof and pharmaceutically acceptable salts thereof.

3) A pharmaceutical composition according to claim 2 wherein the carbamate is physostigmine or a pharmaceutically acceptable salt thereof.

4) A pharmaceutical composition according to claim 3 wherein the carbamate is physostigmine salicylate.

5) A pharmaceutical composition according to claim 1 wherein the first anticholinergic is selected from the group consisting of aprophen, atropine, azaprophen, benactyzine, biperiden, procyclidine, hyoscine and pharmaceutically acceptable salts thereof.

6) A pharmaceutical composition according to claim 5 wherein the first anticholinergic is hyoscine or a pharmaceutically acceptable salt thereof.

7) A pharmaceutical composition according to claim 6 wherein the first anticholinergic is hyoscine hydrobromide.

8) A pharmaceutical composition according to claim 1 wherein the second anticholinergic is selected from the group consisting of aprophen, atropine, azaprophen, benactyzine, biperiden, procyclidine, hyoscine and pharmaceutically acceptable salts thereof characterised in that it is different to the first anticholinergic.

9) A pharmaceutical composition according to claim 8 wherein the second anticholinergic is a pharmaceutically acceptable salt of hyoscine characterised in that it is a different salt to the first anticholinergic.

10) A pharmaceutical composition according to claim 9 wherein the second anticholinergic is hyoscine methyl nitrate.

11) A pharmaceutical composition according to any of the claim 1 wherein the pyridinium salt is a pyridinium oxime or is the deoximinomethyl analogue of such an oxime.

12) A pharmaceutical composition according to claim 11 wherein the pyridinium salt is selected from the group consisting of a compound according to formula I: 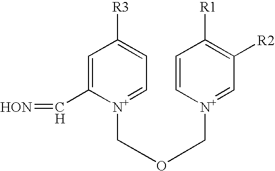
and pharmaceutically acceptable salts thereof, wherein R1 is selected from the group consisting of hydrogen and —CONH₂; wherein R2 is selected from the group consisting of hydrogen, —CONH₂, —COC₆H₅ and —COC₆H₁₁ and wherein R3 is selected from the group consisting of hydrogen and —CHNOH;
a compound according to formula II: 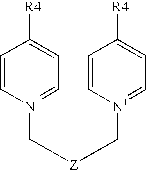
and pharmaceutically acceptable salts thereof, wherein when Z is oxygen, R4 is selected from the group consisting of hydrogen, —CHNOH and —C(CH₃)₃; wherein when Z is —CH₂—, R4 is selected from the group consisting of hydrogen and —CHNOH;
a compound according to formula III: 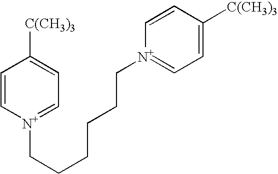
and pharmaceutically acceptable salts thereof;
a compound according to formula IV: 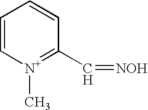
and isomers and pharmaceutically acceptable salts thereof;
and mixtures thereof.

13) A pharmaceutical composition according to claim 12 wherein the pyridinium salt is a pharmaceutically acceptable salt of (1-(2′-hydroxyiminomethyl-1′-pyridinium)-3-(4″carbamoyl-1″-pyridinium)-2-oxapropane).

14) A pharmaceutical composition according to claim 1 wherein the weight ratio of
(i):(ii) is in the range of from about 1:5 to about 1:20;
(i):(iii) is in the range of from about 1:5 to about 1:20;
(i):(iv) is in the range of from about 1:200 to about 1:800;
(ii):(iii) is in the range of from about 1:2 to about 2:1;
(ii):(iv) is in the range of from about 1:20 to about 1:80; and
(iii):(iv) is in the range of from about 1:20 to about 1:80.

15) A pharmeuctical composition according to claim 13 wherein the weight ratio of (i):(ii):(iii):(iv) is about 1:10:10:500

16) A kit comprising
(i) a carbamate;
(ii) a first anticholinergic
(iii) a second anticholinergic and
(iv) a pyridinium salt,
wherein each component is dosed simultaneously, sequentially or separately.

17) A kit according to claim 15 comprising
(i) a pyridinium salt; and
(ii) a pharmaceutical composition comprising a first anticholinergic, a second anticholinergic and a carbamate
wherein (i) and (ii) are dosed simultaneously, sequentially or separately.

18) Use of a pharmaceutical composition comprising
(i) a carbamate;
(ii) a first anticholinergic
(iii) a second anticholinergic and
(iv) a pyridinium salt,
for the manufacture of a medicament for the treatment of poisoning by organophosphates, including nerve agents and pesticides.

19) Use of a pharmaceutical composition according to claim 17 wherein the pharmaceutical composition is administered intramuscularly, intravenously, intranasally, intradermally or orally.

20) Use of a pharmaceutical composition according to claim 18 wherein the pharmaceutical composition is administered intravenously or intramuscularly.

21) Use of a pharmaceutical composition according to claim 19 wherein the pharmaceutical composition is administered intramuscularly.

22) A method for the treatment of organophosphate poisoning in a mammal, including man, comprising administration of an effective amount of a pharmaceutical composition comprising
(i) a carbamate;
(ii) a first anticholinergic
(iii) a second anticholinergic and
(iv) a pyridinium salt.
